(19) United States
(12) Patent Application Publication
(10) Pub. No.: US 2025/0065056 A1
(43) Pub. Date: Feb. 27, 2025

(54) DRUG DELIVERY DEVICE WITH IMPROVED NEEDLE COVER

(71) Applicant: Sanofi, PARIS (FR)

(72) Inventors: Max Davis, Melbourn, Hertfordshire (GB); Thomas Mark Kemp, Melbourn, Hertfordshire (GB); Tom Lever, Melbourn, Hertfordshire (GB); Max Wehebrink, Melbourn, Hertfordshire (GB); Robbie Wilson, Melbourn, Hertfordshire (GB)

(21) Appl. No.: 18/718,756

(22) PCT Filed: Dec. 13, 2022
(86) PCT No.: PCT/EP2022/085637

§ 371 Date: Jun. 11, 2024

(30) Foreign Application Priority Data
Dec. 15, 2021 (EP) 21315275.4

(51) Int. Cl. A61M 5/32

(52) U.S. Cl. CPC A61M 5/3243

(57) ABSTRACT

Disclosed is a drug delivery device including a housing extending along a longitudinal axis of the drug delivery device, and a distal device portion. The distal device portion includes a distal portion, a proximal portion and at least one intermediate portion. The distal portion includes a distal opening allowing passage of a needle and a bearing surface. The intermediate portion includes at least one wall portion extending proximally from the distal portion to the proximal portion. In at least one state of the drug delivery device, a first radial outer extension of the distal portion in a region axially adjoining the bearing surface is smaller than a second radial outer extension in the intermediate portion, and an outer face of the intermediate portion is inclined relative to the longitudinal axis in a cross section along the longitudinal axis.

### CROSS REFERENCE TO RELATED APPLICATIONS

The present application is the national stage entry of International Patent Application No. PCT/EP2022/085637, filed on Dec. 13, 2022, and claims priority to Application No. EP 21315275.4, filed on Dec. 15, 2021, the disclosures of which are incorporated herein by reference.

### TECHNICAL FIELD

The disclosure relates to a drug delivery device with an improved needle cover.

### BACKGROUND

The drug delivery device may be an autoinjector or a manually or semi-automatically operated device. An energy storing element may be used in autoinjectors as well as in semi-automatically operated devices in order to deliver the driving force for the injection operation. The energy storing element may be biased in the factory or by the user prior to use. The drug may comprise insulin or GLP-1 (Glucagon-Like Peptide). However, other drugs may also be injected. Furthermore, other medical devices may also profit from the disclosure, e.g. injectors, spraying devices or inhalation devices.

It may be possible to adapt drug delivery devices to different target groups using needles of different length, i.e. a long needle for adults and a shorter needle for children. However, this may require to have detachable needles or to have different container types with integrated needles. Both options may be cumbersome and/or may have other disadvantages.

### SUMMARY

This disclosure relates to a drug delivery device with enhanced usability and a corresponding method. The device may be easily and/or comfortably to use and/or comprise as few parts as possible. In some embodiments, easy injection into the skin of children is possible.

According to an embodiment, the drug delivery device may comprise:
a housing extending along a longitudinal axis of the drug delivery device, and/or
a distal device portion.

The housing may be e.g. a cylindrical part supporting other parts of the device, e.g. a drive mechanism, a container for a drug, etc. The housing may have a protecting function for the inner parts, e.g. against mechanical impact and/or against environmental influences, e.g. humidity, etc. The distal device portion may be a distal portion of the housing or may be formed by a needle cover that projects distally from the housing, e.g. in at least one operating state of the device.

According to an embodiment, the distal device portion may comprise a distal portion, a proximal portion and at least one intermediate portion. All three portions may be arranged without other portions therein between. Thus, the distal portion may be adjacent to the intermediate portion and the proximal portion may be adjacent to intermediate portion. Alternatively, further portions may be arranged between these portions.

According to an embodiment, the distal portion may comprise a distal opening allowing passage of a needle and a bearing surface. The distal opening may be a needle passage opening through which the needle extents during injection. The needle may not extend to the distal opening before needle insertion into a skin. Thus, the needle may be hide within the distal device portion limiting direct access to the needle and limiting the field of view to the needle.

According to an embodiment, the bearing surface may be configured to be pressed against the skin of a patient during injection of a drug. The bearing surface may be a plane surface. The bearing surface may encompass the distal opening.

According to an embodiment, the intermediate portion may comprise at least one wall portion extending proximally from the distal portion to the proximal portion. The at least one wall portion may comprise an inner surface facing radially inwards and an outer surface or face facing radially outwards. Thin walls may be preferred in order to ease injection molding that may be used to produce the needle cover, e.g. enabling short cooling times and/or preventing deforming of material during cooling.

According to an embodiment, in at least one state of the drug delivery device, a first radial outer extension, e.g. a diameter or a width, of the distal portion in a region axially adjoining the bearing surface may be smaller than a second radial outer extension, e.g. a diameter or a width, in the intermediate portion. The difference in radial extension may be a result of a widening or broadening of the distal portion and/or of the intermediate portion for axial positions with an increasing distance to the axial position of the distal portion. Vice versa, there may be a narrowing of the intermediate portion and/or distal portion in the opposite direction, e.g. for axial positions having a decreased distance to the distal portion, e.g. to the bearing face. There may be a continuous widening/narrowing. Alternatively, a step profile or another profile may be used. The narrow distal portion may allow enhanced usability of the drug delivery device, e.g. more space for pinching of the skin and/or more space for viewing the injection site.

According to an embodiment, an outer face of the intermediate portion may be inclined relative to the longitudinal axis in a cross section along the longitudinal axis A. The inclination may result in different outer extensions, e.g. in the different values of the first radial outer extension and the second radial outer extension. Thus, the difference between the first radial outer extension and the second radial outer extension may corresponds to the amount of an inclination angle of the outer face of the intermediate portion. The inclination may be constant within the intermediate portion, e.g. as is the case for a conical portion. Alternatively, the inclination may change within the intermediate portion depending on the axial position. The inclination may e.g. increase continuously with greater axial distance to the axial position of the distal portion.

According to a further embodiment, the drug delivery device may comprise a retaining space for a drug container or both a retaining space for a drug container and the drug container. The drug container may comprise a needle or a needle coupling element for attaching a needle.

According to a further embodiment, an axial distance between a first axial position on which the first radial outer extension is measured and a second axial position on which the second radial outer extension is measured may have a length of e.g. 3 mm (millimeter), 5 mm and/or of e.g. less than 3 cm (centimeter).

According to an embodiment, in a cross section including or along the longitudinal axis, the outer face of the intermediate portion and the longitudinal axis may include an angle in the range of 15 degrees to 75 degrees, in the range of 30 degrees to 60 degrees or in the range of 40 degrees to 50 degrees. Thus, the inclination may be appropriate to enable the enhanced usability, e.g. closer access to the distal opening (needle passage opening), better line of sight to injection site and/or to pinching of skin. The vertex of the angle may point distally or may lie more distally compared to arms of angle.

According to an embodiment, the distal device portion may be at least a portion of a needle cover coupled to the housing. The needle cover may have several functions, e.g. a trigger function for triggering an injection of the drug, a protection function before and after needle injection, etc.

According to an embodiment, the needle cover may be a part that is manufactured separate from the housing. Thus, the needle cover and the housing may be designed and produced independently from each other, e.g. allowing more degrees of freedom for the design process and/or making production easier compared to production of a single part fulfilling several functions.

According to an embodiment, the needle cover may be movable relative to the housing. Thus, the needle cover may fulfill further function for the operation of the drug delivery device, e.g. a trigger function, a locking function after usage, etc. The needle cover may be moved at least partially into the housing during activation and/or during usage of the device.

According to an embodiment, the needle cover may not have direct mechanical contact to a drug container and/or to a needle of the drug delivery device. Thus, the needle cover is different from e.g. a rigid needle shield RNS that may also cover the needle in order to provide further protection and/or to provide sterility.

The fingers of a user may be placed with a corresponding distance to a movable needle cover and/or to the housing during insertion of the needle and during injection in order to not interfere with the housing during injection of drug.

According to a further embodiment, alternatively, the distal device portion may be a distal portion of the housing. In this case, the fingers of a user may remain in touch with the narrow portion of the distal device portion also during needle insertion and/or injection. Interference of the finger and of movable device parts is not possible as the distal device portion is part of the housing and is not movable relative to the housing.

According to an embodiment, the distal portion and at least a part of the intermediate portion may be accessible on the exterior of the drug delivery device at least in a state before the injection of the drug and/or before insertion of a needle of the drug delivery device. Thus, touching of the distal portion and/or intermediate portion may be possible during preparation of the injection, e.g. pinching of the skin. The intermediate portion may allow a better line of sight before needle injection and when making contact between the bearing surface and the skin.

According to an embodiment, the distal portion may have a rotationally asymmetric outer shape with respect to a rotation around the longitudinal axis. The asymmetry may be used to align a drug viewing window appropriately such that the user may be monitor delivery of drug during injection within this drug window. The asymmetry may have further or other technical effects, e.g. providing a more stable distal device portion, e.g. needle cover.

According to a further embodiment, the proximal portion may have a rotationally symmetric outer shape with respect to a rotation around the longitudinal axis. Thus, manufacturing may be easier compared to the manufacture of asymmetric parts, especially if injection molding is used and corresponding molding tools. Milling may be easier and/or faster for symmetrical parts.

According to an embodiment, the distal opening may have a rotationally asymmetric shape with respect to a rotation around the longitudinal axis. This may allow advanced usability of the device, e.g. better alignment of the device. Furthermore, the same alignment of an asymmetric distal portion and an asymmetric opening may ease manufacturing, e.g. especially if injection molding is used, e.g. the parts may be simpler and/or have thinner walls resulting in shorter cooling times and less deforming during cooling.

According to a further embodiment, the distal opening may have a rotationally symmetric shape with respect to a rotation around the longitudinal axis. Thus, a tool may be produced easier compared to a tool for the production of asymmetric openings.

According to an embodiment, the first radial outer extension and the second radial outer extension may be constant, e.g. may be the same, in all operating states of the drug delivery device. This may simplify the design and/or manufacturing and/or assembling of the device compared to the usage of movable parts as mentioned below.

According to an embodiment, the distal device portion may be a first needle cover or may be comprised in a first needle cover. The drug delivery device may comprise a second needle cover, e.g. a rigid needle shield (RNS). The second needle cover may be removed from the needle before insertion of the needle. Removal of the second needle cover, e.g. of an RNS, through the first needle cover may be possible, e.g. through the distal opening. However, other technical solution may be envisaged, e.g. removal of the second needle cover, e.g. of an RNS, from the syringe shortly before the injection and outside of the housing.

According to an embodiment, the second needle cover may have a maximal outer diameter in a cross section perpendicular to the longitudinal axis. The distal opening may have a first width along the first axis. The first width may be greater than the maximal outer diameter, preferably greater by at most 5 percent of the maximal outer diameter of the second needle cover. Thus, a rigid needle shield may be moved easily through the opening within the first needle cover.

According to a further embodiment, the second needle cover, e.g. RNS, may comprise a flexible elastic boot, e.g. in order to provide sterility and/or to provide protection against mechanical impact. A distal opening in the second needle cover, e.g. RNS, may reduce a proximal force together with the elastic boot, e.g. providing a synergistic effect.

Alternatively, according to an embodiment, the distal device portion may again be a first needle cover or may be comprised in a first needle cover. However, the needle cover may comprise at least two segments, e.g. segments that are pivotable with respect to one another or with respect to the proximal portion. There may be of course more than two segments within the distal device portion, e.g. 3, 4 or 5 segments.

According to an embodiment, a first segment of the at least two segments may comprise a first part of the distal portion and a first part of the intermediate portion. A second segment of the at least two segments may comprise a second part of the distal portion and a second part of the distal portion. Thus, the segments may have a simple form that is easy to manufacture.

According to an embodiment, the first part of the intermediate portion may be pivotable coupled to the proximal portion of the needle cover via a first coupling element. The second part of the intermediate portion may be pivotable coupled to the proximal portion of the needle cover via a second coupling element. Thus, short and simple coupling elements may be used. However, other positions for coupling may be used as well.

According to an embodiment, in an open state of the at least two segments, the first radial outer extension may have a first value, e.g. in order to enable attaching (capping) of a distal cap to the device or in order to fulfill other functions.

According to an embodiment, in a closed state of the at least two segments, the first radial outer extension may have a second value. The second value may be appropriate for needle insertion and/or injection.

According to an embodiment, the first value may be greater than the second value by at least 20 percent or by at least 50 percent of the second value. This may allow easy removal of a second needle cover, e.g. of an RNS in the first state and providing a narrow opening in the second state and/or allowing close access to the needle passage opening in the second state.

According to a further embodiment, the segments may comprise a rigid material, e.g. a material having no resiliency or only slight resiliency, e.g. if compared with elastic material as mentioned below.

According to an embodiment, the drug delivery device, preferably the needle cover, may comprise at least one resilient element that biases the at least two segments to pivot into the closed state. These resilient elements may be different from coupling elements between the intermediate portion(s) and the proximal portion to give only one example. Thus, the resilient elements may be easily adapted to its main function.

According to an embodiment, the at least one resilient element may be attached to the first segment and to the second segment. Thus, it may be possible to reduce the number of resilient elements as each resilient element acts a force to both segments. Furthermore, the leverage effect may be used if the resilient elements are arranged on a middle part of the intermediate portion(s) or more distally.

According to an embodiment, the at least one resilient element may be or may comprise a tension element that applies a tension force to the at least two segments. Usage of a tension force may be simpler compared to usage of other forces, e.g. of a compression force. However, in other embodiment other forces may be used instead or in addition to a tension force in order to bias the segments.

According to an embodiment, the at least one resilient element may be or may comprise an elastic band or a tension spring. Thus, a simple mechanical element may be used.

According to a further embodiment, other resilient elements may also be used, e.g. springs comprising at least one winding or a plurality of windings, e.g. 2, 3, 4, etc. windings but e.g. less than 100 windings. Springs made of a metal may be used, e.g. made of steel.

The at least one resilient element may comprise or may be an elastic band, a synthetic material and/or synthetic rubber or natural rubber, e.g. caoutchouc. An elastomer, silicone, silicone elastomer, etc. may be used as well.

The at least one resilient element may be adhered to the respective segment, e.g. by gluing or adhesive bonding. Alternatively, other coupling technologies may be used, e.g. fusing, welding, etc. Usage of two component molding may be advantageous, e.g. with regard to production costs and/or simplicity

According to an embodiment, the at least one resilient element may be arranged inside the needle cover. Thus, the resilient element may be protected by the segments and/or may not be visible for the user.

Alternatively, it is of course possible to arrange the at least one resilient element outside on an outer face (surface of the needle cover) of the segments, e.g. within a circumferential groove in order to provide safe seat or fit.

According to an embodiment, the first coupling element and/or the second coupling element may be formed integrally with the proximal portion of the needle cover, preferably as a film hinge or comprising a film hinge. Thus, production effort and/or assembling effort may be reduced.

The coupling elements may have no resiliency. Alternatively, the coupling elements may have resiliency, e.g. in order to bias the segments to the close position. In this case, further resilient elements as mentioned above may be used in addition to the resilient coupling elements. Alternatively, no further resilient elements may be necessary in addition to resilient coupling elements.

According to an embodiment, the needle cover may be a first needle cover. The drug delivery device may comprise a second needle cover. In the open state, at least one of the first section and the second section may abut to the second needle cover or to an insert of a cap that holds the second needle cover. Thus, easy attachment of a cap may be possible.

According to an embodiment, the second needle cover may have a maximal outer diameter in a cross section perpendicular to the longitudinal axis. The distal opening may have a first width along the first axis. In the open state, the first width may be greater than the maximal outer diameter, preferably greater by at most 10 percent or by at most 5 percent of the maximal outer diameter of the second needle cover. The value 10 percent may be valid if the insert is used. Thus, the first width may be made as small as possible in order to reduce force within the resilient elements.

According to a further embodiment, there may be an intermediate state between the open state and the closed state. The intermediate state may be a state of the device in which the cap and a second needle cover, e.g. an RNS, is or are partially removed from the drug delivery device. At least one of the first section or the second section may abut to the second needle cover, e.g. RNS, but not the insert anymore. The first width may reduce from the open state to the intermediate state and then to the closed state of at least two sections. In the closed state of at least two section there may be a width of the distal opening that is still greater 0 (zero) mm (millimeter) in order to enable that needle may extend through distal opening.

Further alternatively, according to an embodiment, at least one of the intermediate portion and the distal portion may comprise an elastic material. An elastic material may return after releasing of a load to the undeformed initial state which was present before application of the load. The amount of elasticity may be chosen appropriately. The needle cover comprising the elastic material may be produced by injection molding and/or may provide elasticity without using windings as known from springs. The needle cover comprising the elastic material may be an integral part and/or a single part, e.g. simplifying design and/or production and/or assembling of the device.

According to an embodiment, the elastic material may be configured to allow decreasing of the first radial outer extension, e.g. if a second needle cover, e.g. an RNS, is removed from the device.

According to an embodiment, in an open state of the distal opening, the first radial outer extension may have a first value. Thus, the elastic material may be in an expanded state.

According to an embodiment, in a narrow state of the distal opening, the first radial outer extension may have a second value. Thus, the elastic material may be in a completely relaxed state or in a more relaxed state compared to the expanded state.

According to an embodiment, the first value may be greater than the second value by at least 20 percent or by at least 50 percent of the second value. Thus, removal of a comparably broad second needle cover may be possible as well as providing a narrow distal opening after removal of the second needle cover through an opening of the distal device portion, e.g. through the distal opening. The narrow distal opening may allow a narrow distal portion, e.g. providing all the technical effects mentioned above, e.g. with respect to the line of sight and/or with respect to access to the injection site.

The second radial outer extension that is measured at the intermediate portion may also decrease if the needle cover transitions from the first state (open distal opening) to the second state (narrow distal opening). Thus, the elastic material may allow lateral movement of at least a portion of the outer face of the intermediate portion. This may ease narrowing of the distal portion and/or enable better line of sight and/or better access to the injection site.

The elastic material may comprise an elastomer, a silicone, a silicone elastomer, etc. Other elastic materials may be used as well. The needle cover may be completely made of an elastic material. Alternatively, only the intermediate portion and or the distal portion may be made of or may comprise an elastic material. In this case, connection to the proximal portion may be made in several ways, e.g. adhering techniques may be used, e.g. gluing or adhesive bonding, or other connection techniques, e.g. fusing welding or molding, e.g. two component molding. However, other mechanical connection techniques may be appropriate, e.g. form fit and/or force fit. The elastic portion(s) of the needle cover may be imposed on a rigid portion, e.g. using protrusion or a circumferential rim in order to provide a good coupling.

Thus the elastic material may fulfill several functions, e.g. a first function that provides tension or a tension force and a second function that is similar to function of a hinge or of the hinge(s) as mentioned above. There may be a synergistic effect of both functions, e.g. narrowing of distal opening is eased by shrinking the lumen that is encompassed by the intermediate portion.

Compared to the material of the distal portion and/or of the intermediate portion, the proximal portion may be made of the same material, e.g. formed integrally, or may be made of a more rigid material, e.g. in order to enable long arms extending proximally. The intermediate portion may be coupled via the proximal portion to other parts of the needle cover and/or to the housing, e.g. by form fit and/or by impose it on more proximal part of needle cover.

According to an embodiment, the distal device portion may be a first needle cover or may comprised a first needle cover. The drug delivery device may comprise a second needle cover. In the open state, the elastic distal portion may abut to the second needle cover or to an insert of a cap of the drug delivery device. The insert of the cap may support and/or hold the second needle cover. A narrow state of the distal opening may be activated if the cap and/or the second needle cover, e.g. an RNS, is or are removed. Optionally, the cap may comprise the insert. Thus, easy removal of the second needle cover prior to injection is possible due to the usage of the elastic material.

According to an embodiment, the second needle cover RNS may have a maximal outer diameter in a cross section perpendicular to the longitudinal axis. The distal opening may have a first width along the first axis. In the open state, the first width may be equal to or may be greater than the maximal outer diameter, preferably greater by at most 10 percent or by at most 5 percent of the maximal outer diameter of the second needle cover. The value of 10 percent may be valid if the insert is used that is mentioned above and below.

Again, there may be an intermediate state between the open state and closed state, when the cap and the second needle cover, e.g. an RNS, are partially removed from the drug delivery device and at least one inner edge of the elastic distal portion abuts to the second needle cover, e.g. an RNS, and not to the insert any more. The first width may reduce from the open state to the intermediate state and finally to the narrow state of the distal portion and/or of the distal opening. In the narrow state the width of the distal opening may be still greater 0 (zero) mm (millimeter) in order to enable that needle may extend through the distal opening.

Thus, in the open state at least one inner edge of the elastic distal portion may abut to the second needle cover, e.g. an RNS, or to the insert of the cap for holding the second needle cover. This may allow usage of appropriate cap designs.

According to an embodiment, the drug delivery device may comprise at least one compression spring. The at least one compression spring may be configured to bias the needle cover distally, wherein the needle cover may comprise the distal device portion. This may assist the functions of the needle cover mentioned above, e.g. coverage of the needle before and after injection, e.g. in order to reduce the danger of accidental needle sticks.

The distal end of the compression spring may be coupled to a distal portion of the needle cover (first needle cover, outer needle cover in case that there is more than one needle cover), e.g. to inner circumferential rim or ledge of the needle cover. The proximal end of the compression spring may be coupled to the housing. More than one compression spring may be used.

According to a further embodiment, the biasing of the needle cover may be used within an autoinjector. Activation of a driving mechanism may be initiated or enabled via the distally biased needle cover. The autoinjector may comprise a drive spring, e.g. a further compression spring that is adapted and/or configured to drive a piston rod into a medicament container.

According to further embodiment, for an autoinjector, other designs principles are also possible that do not use distal biasing of the distal device portion, e.g. usage of a movable cartridge allowing automatic needle insertion and/or automatic needle retraction.

According to an embodiment, the drug delivery device may comprise a cap. The cap may be rotationally asymmetric with respect to the longitudinal axis in order to adapt the cap to a rotational asymmetry of the distal device portion. The cap may be adapted to a rotational asymmetry of at least one of:
The shape of the distal opening, e.g. to at least one of the asymmetries mentioned above,
An outer shape of the distal portion, e.g. to at least one of the asymmetries mentioned above,
An outer shape of the intermediate portion, e.g. to at least one of the asymmetries mentioned above, and/or
An inner shape of the intermediate portion, e.g. to at least one of the asymmetries mentioned above.

The adaption of the cap may be made e.g. in order to:
Save material/plastic, and/or
Ease manufacturing and/or assembling of the drug delivery device, and/or
Ease removal of the cap.

According to a further embodiment, the cap may be adapted to the outer shape of the intermediate portion.

According to a further aspect and embodiment that is not yet claimed, a method of using a drug delivery device according to any one of the embodiments mentioned above is provided comprising the following:
The drug delivery device is held with a first hand during injection of the drug,
Another hand, preferably of the same person to which the first hand belongs, is used to pinch the skin of the patient, preferably the skin of a child or of a baby during injection, and
The inclined face of the intermediate portion enables a greater field of view to the injection site.

Reduced injection depth into the skin is possible due to pinching and/or due to the usage of the distal device portion, e.g. needle cover, mentioned above. A narrow portion of distal device portion, e.g. needle cover, may enable and/or ease pinching of the skin. Thus, it may be prevented that the drug is accidentally injected into muscles and/or bones.

Manipulation of the skin may be easier due to inclined face of intermediate portion and/or the narrow distal portion, e.g. in all lateral directions or in only some of the lateral directions of the distal portion.

The skin that is pierced by the needle may be the skin of child, e.g. of a person of less than 14 years, of less than 10 years, or even of less than 5 years. Alternatively, the skin may be the skin of a baby, e.g. having an age of less than 1 year.

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference numerals refer to same technical features if not stated otherwise. As far as “may” is used in this application it means the possibility of doing so as well as the actual technical implementation. The present concepts of the present disclosure will be described with respect to preferred embodiments below in a more specific context namely drug delivery devices, especially drug delivery devices for humans or animals. The disclosed concepts may also be applied, however, to other situations and/or arrangements as well, e.g. for other injectors, spraying devices or inhalation devices.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

### BRIEF DESCRIPTION OF THE FIGURES

For a more complete understanding of the presently disclosed concepts and the advantages thereof, reference is now made to the following description in conjunction with the accompanying drawings. The drawings are not drawn to scale. In the drawings the following is shown in:

FIGS. 1a to 1D a cross section of a drug delivery device according to a first embodiment and in different operating states,

FIG. 2 a drug delivery device according to a second embodiment comprising a needle cover with a slit like opening,

FIG. 3 a closer view of the drug delivery device according to FIG. 2,

FIG. 4 a cross section of an application example of the drug delivery device according to FIG. 2,

FIG. 5A a perspective view of a drug delivery device according to a third embodiment,

FIG. 5B an axial view of the drug delivery device according to FIG. 5A,

FIG. 6A a perspective view of a drug delivery device according to a fourth embodiment with an open distal aperture,

FIG. 6B a perspective view of the drug delivery device according to FIG. 6A a with a closed distal aperture,

FIG. 7A a longitudinal cross section of a drug delivery device according to a fifth embodiment with an open distal aperture and a cap and a rigid needle shield in place,

FIG. 7B a longitudinal cross section of the drug delivery device according to FIG. 7A during removal of the cap and of the rigid needle shield,

FIG. 7C a longitudinal cross section of the drug delivery device according to FIG. 7A with the cap and the rigid needle shield removed,

FIG. 8A a perspective view of a drug delivery device according to a sixth embodiment with an open distal aperture of a flexible and resilient needle cover, and

FIG. 8B a perspective view of the drug delivery device according to FIG. 8A a with a narrow distal aperture.

### DETAILED DESCRIPTION

Reference may be made to a cylindrical coordinate system, i.e. each position may be defined by three coordinates: axial value (height, distance to zero plane), radial distance to axis and angle between current radial position and a plane that is defined as having angle zero. In this document the words “in an axial position” may mean having an axial coordinate.

The distal end D may be an end that is closer to a needle compared to a proximal end P.

Reference signs having the same last two digits may refer to same elements if not mentioned otherwise, e.g. needle cover 208 and 308.

Certain embodiments in this document are illustrated with respect to an injection device comprising an advanced needle cover used as an activation element, e.g. an autoinjector. Reference is made to WO 2015/004052 A1 in this regard which is included by reference for all legal purposes.

However, other embodiments comprising the advanced needle cover may relate to drug delivery devices comprising other activation mechanism or operated by a manual driving force. Reference is made e.g. to WO 2014/033195 A1 or to WO 2014/033197 A1 in this regard which are included by reference for all legal purposes. The injection button may provide at least one user interface member for initiating and/or performing a dose delivery operation of the drug delivery device. The (dial) grip or knob may provide a user interface member for initiating and/or performing a dose setting operation using a dose setting surface, e.g. the circumferential surface of the (dial) grip or knob. A delivery surface may be used to initiate dose delivery. The delivery surface may be the proximal P surface of the (dial) grip or knob.

The device may be of the dial extension type, i.e. its length may increase during dose setting or dose dialing. Other injection devices with the same kinematical characteristic of the dial extension and button during dose setting and dose expelling operational mode are known as, for example, Kwikpen® and Savvio® device marketed by Eli Lilly as well as FlexPen®, FlexTouch® and Novopen® 4 device marketed by Novo Nordisk or devices of other manufacturers. An application of the general principles disclosed herein to these devices therefore appears straightforward and further explanations will be omitted. Alternatively, the proposed concepts may be used in devices that are not of the dial extension type but include for instance torsion spring that are biased by rotation of a dial knob. Moreover, fully mechanically driven or electromechanically driven drug delivery devices may be used, e.g. comprising an electrical motor. A distance sleeve may be used in order to have a reference injection depth that may be adjusted using the spacer units, e.g. in case that other actuating elements than a movable needle cover (needle protection element) are used.

However, the general principles of the present disclosure are not limited to that kinematical characteristic. Certain other embodiments may be conceived, e.g. for application to other injection devices of Sanofi or devices of other manufacturers where there is a container for a drug that may be moved axially relative to a housing of the drug delivery device in order to insert and/or to retract a needle.

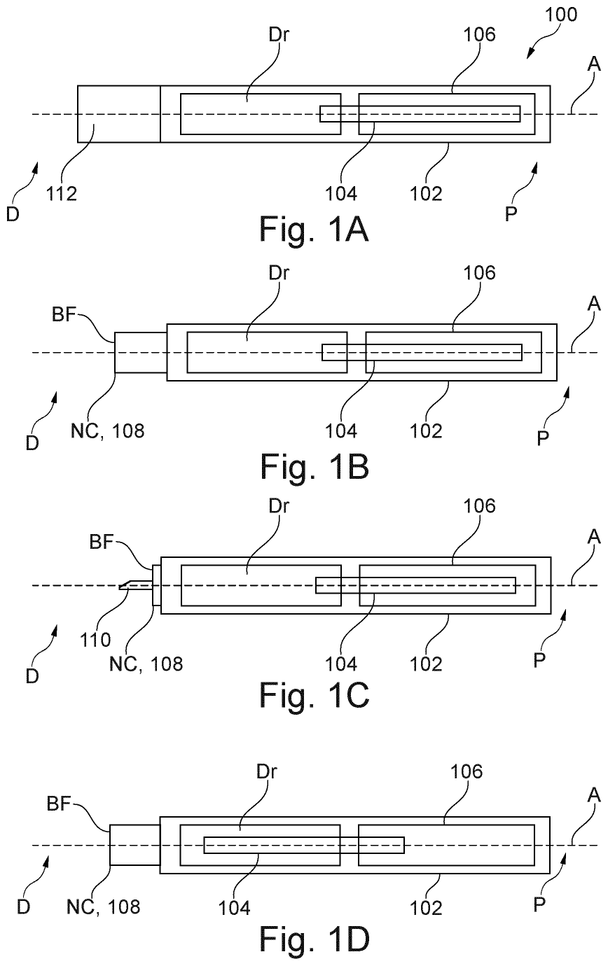

FIGS. 1A through 1D illustrate an embodiment of a drug delivery device 100. Device 100 may be suitable as the device in the drug delivery arrangements described further above and below. The figures show device 100 in different states during its operation.

FIG. 1A illustrates drug delivery device 100 in an initial or as delivered state. Drug delivery device 100 may comprise a housing 102. Housing 102 may be provided to retain and/or may retain a medicament container in its interior. Medicament, e.g. liquid medicament or drug Dr, may be arranged in the medicament container. Housing 102 be provided to retain and/or may retain a needle 110, see FIG. 1C. In other words, needle 110 may be arranged or may be arrangeable in housing 102. Needle 110 can be an integral part of the medicament container, e.g. (permanently or releasably) connected to a medicament container body, or separate from the medicament container. In the first case, the medicament container may be a syringe. In the second case, the medicament container may be a cartridge. In case a cartridge is used as medicament container, initially, the medicament container and the needle can be fluidly disconnected and fluid communication between the medicament container interior and the needle may only established during operation of drug delivery device 100.

A drive mechanism 106 provided to drive a drug delivery operation may expediently be provided in the housing 102. Drive mechanism 106 may comprise a plunger rod 104. Drug delivery device 100 may further comprise a drive energy source, e.g. a drive spring, such as a compression spring, (not explicitly shown). The drive energy source may be arranged to drive plunger rod 104 in a distal direction D relative to the medicament container during the drug delivery operation. During this movement, a stopper, which may be movably retained in the medicament container and may seal the medicament container proximally, may be displaced towards an outlet of the medicament container to dispense drug Dr or medicament retained within the medicament container through the outlet. The outlet may be formed or defined by needle 110, see FIG. 1C.

Other potential drive energy sources different from a spring comprise an electrical power cell or battery for driving plunger rod 104 by a motor or a reservoir suitable to provide gas pressure, where the gas pressure can be used to drive the drug delivery operation.

Drug delivery device 100 may be an autoinjector. The energy for driving the drug delivery operation in an autoinjector may be provided by components integral to drug delivery device 100 and does not have to be loaded into the device by the user during the operation of device 100 as is the case in many spring driven pen-type variable dose injectors, where, usually, the energy is loaded into the spring by the user during a dose setting procedure.

Drug delivery device 100 may expediently be a single shot device, i.e. it is provided to dispense only one dose. Drug delivery device 100 may be a disposable drug delivery device 100, that is to say a device 100 which is disposed of after its use. Device 100 may be a pen-type device. The medicament container and/or needle 110 may be axially secured within drug delivery device 100, e.g. within housing 102, or may be movable relative to housing 102, e.g. for piercing the skin. In the first case, the user may have to perform the movement for piercing the skin with needle 110. In the second case, piercing of the skin by needle 110 may be driven by a needle insertion mechanism of the drug delivery device. Automatic needle retraction may be used as well.

As depicted in FIG. 1A, drug delivery device 100 may further comprises a cap 112. Cap 112 may be arranged at the distal end D of drug delivery device 100. Cap 112 may be detachably connected to the remainder of device 100, e.g. to housing 102 and/or another component or member of drug delivery device 100. Cap 112 may cover a distal end D of the remainder of drug delivery device 100 and/or a needle passage opening through which needle 110, e.g. the distal needle tip, may pass to pierce the skin from the interior of drug delivery device 100 during or for the drug delivery operation. Cap 112 may comprise a needle shield remover, which may engage a rigid needle shield RNS, which may cover needle 110 such that the rigid needle shield is removed from needle 100 together with cap 112, e.g. when cap 112 is detached or disconnected from device 100. Housing 102 may expediently cover the majority of the length of drug delivery device 100, e.g. 60 percent or 70 percent or more percent of the entire length of the drug delivery device 100 (with the cap 12 attached and/or with the cap detached).

FIG. 1B illustrates drug delivery device 100 with cap 112 being removed. According to FIG. 1B, device 100 may be in a state ready to be operated, e.g. ready to perform a drug delivery operation when the operation is triggered. As depicted, drug delivery device 100 may further comprises a needle cover NC. Needle cover NC may protrude distally from housing 102 and/or may have been covered by the cap 112 when cap 112 was still attached to housing 102. Needle cover NC may be movable relative to the housing 10 from an initial position or first position to a second position or trigger position. Needle cover NC may be provided to extend beyond the distal tip of needle 110 which may protrude from the housing 102 before the drug delivery operation is commenced. Needle cover NC may be movable in the proximal direction relative to housing 102. During this movement, e.g. before needle cover NC reaches the second position, needle 110 may pierce the skin of the user.

Needle cover NC may serve as a trigger member 108 of the drug delivery device. Needle cover NC as trigger member 108, when displaced proximally from the initial or first position depicted in FIG. 1B to the second or trigger position (see FIG. 1C), may automatically initialize the drug delivery operation, preferably when it is in the second position. The drug delivery operation can be initialized by removing a mechanical lock which prevents movement of plunger rod 104 in the distal direction or by moving plunger rod 104 to disengage a mechanical lock via moving needle cover NC. Alternatively, needle cover NC when moved from the first position to the second position and expediently when in the second position may only enable triggering of the drug delivery operation. In this case, a separate trigger member, e.g. a trigger button on the proximal end of the housing 102, may be provided to initiate the drug delivery operation. Operating the trigger button to initiate the drug delivery operation may only be possible when needle cover NC is in the second position. In yet another alternative, needle cover NC may only be provided to prevent needle stick injuries before and/or after use of drug delivery device 100. In this case, needle cover NC may be completely decoupled from drive mechanism 106 and/or not may be involved in triggering or enabling triggering of the drug delivery operation at all.

Needle cover NC may be provided to bear against the skin of a user during injection. Hence, the distal surface of needle cover NC may provide a bearing surface or face BF. Bearing surface BF may delimit and/or extend around a needle passage opening provided in needle cover NC. The bearing surface 16 may be ring-like, oval, elliptic, rectangular, quadratic, etc., circumferentially closed and/or be defined by an inward protrusion protruding radially from an inner wall of needle cover NC, e.g. a distal cylindrical portion thereof. Bearing surface BF may be expediently the distal end surface of needle cover NC, e.g. facing distally.

FIG. 1C illustrates needle cover NC in the second position relative to housing 102. This is the position when the drug delivery operation has been initiated, can be initiated, and/or when the needle pierces the skin, for example. Needle 110 may protrude axially from bearing surface BF of drug delivery device 100 (particularly through the needle passage opening in needle cover NC) and, by the distance with which it protrudes over the bearing surface BF, penetrate the skin (the skin is not shown in this representation). This distance may be characteristic for or be equal to the injection depth. The device may be maintained in contact with the skin until the drug delivery operation of drug Dr has been completed, which may be indicated by an optional audible, tactile, and/or visual indication provided by the drug delivery device 100.

After the drug delivery operation has been completed, e.g. piston rod 104 has moved distally, device 100 may be removed from the skin (see FIG. 1D). Needle cover NC may be biased relative to housing 102 towards the first position by a spring (not shown). Thus, when device 100 is removed from the skin needle cover NC may be moved towards the first position with respect to the housing 102. Needle cover NC may be moved distally, e.g. beyond its first position, into a final, third or locked position relative to housing 102. In this position needle cover NC may expediently axially be locked relative to the housing 102 against movement in the proximal direction P, e.g. by a locking engagement between a locking feature of needle cover NC and housing 102. As it is axially locked, needle cover NC may no longer be displaced proximally relative to housing 102 into the second position and/or into the first position. This may protect the user from needle stick injuries after use. In this state, device 100 may be locked, see FIG. 1D.

Alternatively, there may be no movable needle cover. Thus a distal device portion DDP may be pressed against the skin of a user. Needle injection may be performed by a needle insertion mechanism. An optional needle retraction mechanism may also be used. Triggering may be performed using a proximal trigger element, e.g. a proximal knob of modified device 100.

Drug delivery device 100 may comprise an electronic unit that may be mechanically connected to a proximal end region P of drug delivery device 100, for instance to a proximal end region P of device 100. The electronic unit may be used not only for drug delivery device 100 but also for other drug delivery devices that are similar or identical to drug delivery device 100. Alternatively, the electronic unit may be an integrated part of the drug delivery device 100. The electronic unit may be used to monitor drug delivery, e.g. amount of dose, time and date.

The terms “drug” or “medicament” are used synonymously herein and describe a pharmaceutical formulation containing one or more active pharmaceutical ingredients (API) or pharmaceutically acceptable salts or solvates thereof, and optionally a pharmaceutically acceptable carrier. An active pharmaceutical ingredient (API), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug Dr or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug Dr or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2years. Storage may occur at room temperature (e.g., about 20° C.), or refrigerated temperatures (e.g., from about −4° C. (minus 4° C.) to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F (ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

One of the basic concepts proposed in this application is to use a needle cover NC that has an increasing radius for axial positions of needle cover NC that are located more proximally than other axial positions of needle cover NC. Thus, there may be space, e.g. for pinching of the skin and/or for viewing the injection site at the distal end D of drug delivery device 100 or other devices 200 to 800 as mentioned below.

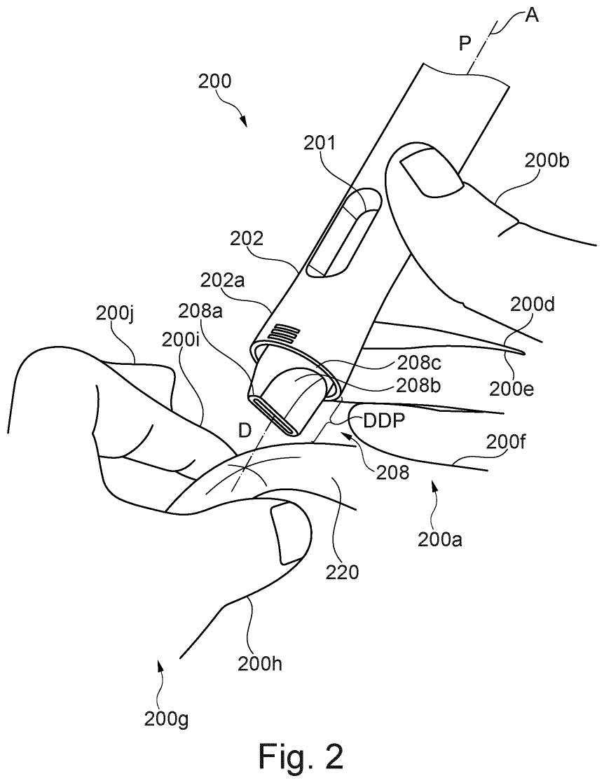

Detailed embodiments of the advanced needle cover NC are described in the following FIGS. 2 to 8B. Other embodiments are of course also possible.

FIG. 2 illustrates a drug delivery device 200 according to a second embodiment comprising a distal device portion DDP. In the second embodiment distal device portion DDP is part of a needle cover 208 with a slit like opening 208, e.g. the length of the slit is essentially longer than the width of the slit, e.g. by at least factor two or at least factor 3. Alternatively, needle cover 208 may have the same outer shape but a rotationally symmetric opening 208, e.g. a circular opening. In other embodiments a distal device portion having the same shape as illustrated in FIG. 2 may be part of housing 202, e.g. there may be other triggering elements than a movable needle cover 208, N.

Drug delivery device 200 may be hold by a right hand 200a, e.g. of child or of an adult who administers drug Dr to a child. Right hand 200a comprises a thumb 200b, an index finger (not shown), a middle finger 200d, a ring finger 200e and a little finger 200f. The or a left hand 200g, e.g. of the child or of the adult who administers drug Dr to the child) comprises a thumb 200h, an index finger 200i, a middle finger 200j and further fingers that are not illustrated. Thumb 200h and index finger 200i may pinch the skin 220 of the child at the injection side, e.g. in order to prevent that the needle touches muscles and/or bones of the child. Unpinching of the skin may occur after injection of drug Dr.

Device 200 may be an autoinjector triggered by a proximal movement of needle cover 208 relative to a housing 202. Alternatively, device 200 may be an autoinjector that is triggered at its proximal end, e.g. comprising a movable container/syringe, for automatic needle insertion and/or retraction. In this case, needle cover 208 may not be movable with respect to the housing 202.

Further, alternatively, device 200 may be manually driven device, e.g. a device wherein needle cover 208 covers the needle (not shown in FIG. 2, see needle 210 in FIG. 4) only partially in an inactivated state of device 200. Also in this case, needle cover 208 may be not movable with respect to the housing 202. The design may be compatible with a manually driven device which may be activated by a button at the proximal end for example.

Device 200 may comprise at least one optional drug window 201 or at least two optional drug windows arranged in the main housing part 202. Drug window 201 may enable to monitor delivery of drug Dr. Main housing part 200 may comprise a distal portion 202a at its distal D end.

Needle cover (e.g. actuating element) 208 may comprise a distal portion 208a, DP. Distal portion 208a, DP of needle cover 208 (needle shroud) may be positioned outside of housing 202. An intermediate portion 208b may also be arranged outside of housing 202, especially in a state in which no injection takes place. A ring portion 208c or proximal portion PP of needle cover 208, NC, may also be arranged outside of housing 202. However, alternatively, ring portion 208c or proximal portion PP may be encompassed by housing 202, especially by distal portion 202a of housing 202. There may be an elongated proximal portion 208c that extends within housing 202, e.g. comprising two proximally extending arms. Alternatively, proximal portion 208c may be comparably short, e.g. not extending proximally to axial positions at the distal end of optional drug window 201.

Furthermore, device 200 may comprise all or some of the parts mentioned above for drug delivery device 100, e.g. a piston rod, a drive mechanism, etc.

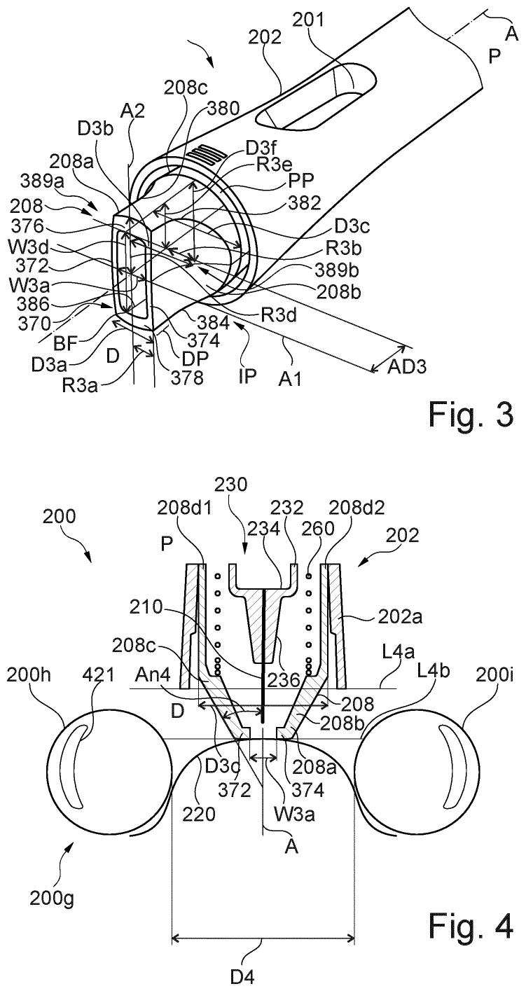

FIG. 3 illustrates a closer view of drug delivery device 200. As is clearly visible needle shroud 208 may have a comparable narrow distal portion 208a, DP, an intermediate portion 208b, IP, e.g. that becomes wider at least on its outside with increased distance to the distal portion DP, e.g. only within some planes comprising longitudinal axis A or within all planes comprising longitudinal axis A, and a ring portion 208c or proximal portion PP that may have a circular outer diameter and does that may not become wider with increased distance to the distal portion DP. In the embodiment, the needle shroud/cover 208 may become wider only in the plane comprising an axis A1 and longitudinal axis A of device 200. Contrary, the needle shroud 208 has essentially the same width or only a slightly increasing width, e.g. less than 5 percent increase of the outer width at the distal end of needle shroud 208, in a plane comprising an axis A2 that is perpendicular to axis A1 and also comprising longitudinal axis A. However, in other embodiments, needle shroud 208 may have an increasing outer width in both planes, e.g. a conical shape may be used.

In the second embodiment, portions 208a, 208b and 208c are not movable relative to each other, e.g. needle cover 208 (shroud) is a part having no parts movable relative to other parts of needle cover 208 and/or no elastically deformable portions or parts.

Distal portion 208a, DP may comprise a distally facing bearing face BF (bearing surface) and may comprise an opening 370. Opening 370 may be formed by:
A long first side wall 372, e.g. a straight side wall 372 that may be parallel to transversal axis A2,
A long second side wall 374, e.g. a straight side wall 374 that may also be parallel to transversal axis A2,
A short third side wall 376 if its length is compared with the length of side wall 372. Side wall 376 may be a straight side wall 374 that may be parallel to transversal axis A1, and
A short fourth side wall 378 if its length is compared with the length of side wall 372. Side wall 378 may be a straight side wall 378 that may be parallel to transversal axis A1.

The following axially directed edges may extend proximally from distal portion 208a, DP to proximal portion 208c, PP:
An “upper” “left” edge 380,
An “upper” “right” edge 382,
A “lower” “right” edge 384, and
A “lower” “left” edge 386, see dashed line.

The positions “upper”, “lower”, “left” and “right” are valid for the view of drug delivery device 200 as illustrated in FIG. 3. However, if device 200 is viewed from another perspective and/or in a different position, these position change correspondingly.

Edges 380 and 386 may come together on proximal portion 208c at the side of device 200 that is hidden in FIG. 3. Edges 382 and 384 may come together on proximal portion 208c as is visible in FIG. 3.

A first concave side face 389a may be formed on the hidden side of device 200 between edges 380 and 386 creating an intermediate space for fingers or for a finger used for pressing and/or pinching skin 220, e.g. for index finger 200i. A second concave side face 389b and a corresponding intermediate space for a finger pressing and/or pinching skin, e.g. thumb 200h, may be formed between edges 382 and 384 as is illustrated in FIG. 3.

There may be the following dimensions in a plane comprising axis A1 and longitudinal axis A:
A radial distance R3a of a radially facing outer face of distal portion 208a, DP from longitudinal axis A. Radial distance R3a is measured along axis A1,
A radial distance R3b of a radially facing outer face of intermediate portion 208b, IP from longitudinal axis A. Radial distance R3b may be measured along an axis that is parallel to axis A1.
An axial distance AD3 between the positions on longitudinal axis A that are used for measuring radial distance R3a and radial distance R3b. Axial distance AD3 may be greater than 3 mm or greater than 5 mm.
A diameter D3c of proximal portion 208c, PP, e.g. parallel to axis A1, and
A width W3a of distal opening 370, e.g. along axis A1.

Distance R3b may be greater than distance R3a, e.g. greater by at least 20 percent or by at least 50 percent of distance R3a. Diameter D3c may be e.g. greater by at least 20 percent or by at least 50 percent of distance R3b, e.g. greater than twice of the distance R3b. All dimensions R3a, R3b, AD3, D3c and W3a may have a fix (constant) value in all operating states of device 200.

There may be the following dimensions in a plane comprising axis A2 and longitudinal axis A:
A radial distance R3d of a radially facing outer face of distal portion 208a, DP from longitudinal axis A and along axis A2,
A radial distance R3e of a radially facing outer face of intermediate portion 208b, IP from longitudinal axis A. Radial distance R3e may be measured along and that may be parallel to axis A2,
The axial distance AD3 between the positions on longitudinal axis A that are used for measuring radial distance R3d and radial distance R3e,
A diameter D3f of proximal portion 208c, PP, e.g. parallel to axis A2, and
A width W3d of distal opening, e.g. along axis A2.

Distance R3d may be equal to distance R3e or there may be only a small difference between both distances, e.g. a difference smaller than 5 percent of distance R3d. Diameter D3f may be equal to twice the distance R3e or there may be only a small difference between diameter D3f and twice the distance R3e, e.g. a difference smaller than 5 percent of distance R3e. Diameter D3e may be equal to diameter D3f, e.g. proximal portion 208c may be a circular portion or a cylindrical portion. As already mentioned, opening 370 may be a slit, e.g. width W3d may be greater than with W3b, e.g. by at least factor 2, 2.5 or 3. In the same way distance R3d may be greater than distance R3a, e.g. by at least factor 2, 2.5 or 3.

Again, all dimensions R3d, R3e, AD3, D3f and W3a may have a fix (constant) value in all operating states of device 200.

Radial outer extensions D3a and D3b may be used instead of distances R3a and R3b. Radial outer extensions D3a is twice the radial distance R3a. Radial outer extensions D3b is twice the radial distance R3b.

Drug window(s) 201 may be aligned to the short sides of opening 370 and/or to the short sides of distal portion 208a, DP in order to allow appropriate preparation of the injection site by the user, e.g. using a free line of sight due to e.g. concave portions 389a or 389b and/or good monitoring of injection of drug Dr through one of the drug windows, e.g. drug window 201. The same may be true for all other embodiments mentioned in this description.

FIG. 4 illustrates a longitudinal cross section of an application example of drug delivery device 200. Device 200 may be in a pre-injection state in which it is still non-activated. Distal part 202a of main housing 202 may encompass the distal part of a syringe 230. Syringe 230 may comprise, a barrel portion 232, a shoulder 234, a neck portion 236 and a flange (not shown). A needle 210 may be integral to neck portion 236, e.g. glued into a central bore of neck portion 236.

As is illustrated in FIG. 4, distal tip of needle 210 may be arranged at an axial position that is encompassed by intermediate portion 208b, IP. Needle 210 may not be arranged within opening 370 in the state illustrated in FIG. 4. Thus, touching of the tip of needle 210 may not be possible. Injuries due to needle sticks may be prevented effectively. Furthermore, needle 210 may not be visible thereby reducing patient's fear of needles.

There may be an optional first arm 208d1 of needle cover 208 (needle shroud). Arm 208d1 may extend proximally, e.g. from ring portion 208c. Furthermore, there may be a second arm 208d2 of needle cover 208 (needle shroud). Arm 208d1 may also extend proximally, e.g. from ring portion 208c. Alternatively, only one arm 208d1, 208d2 may be used for triggering of device 200. In other embodiments of device 200, the device is not triggered using needle cover 208, e.g. arms 208d1 and 208d2 are omitted.

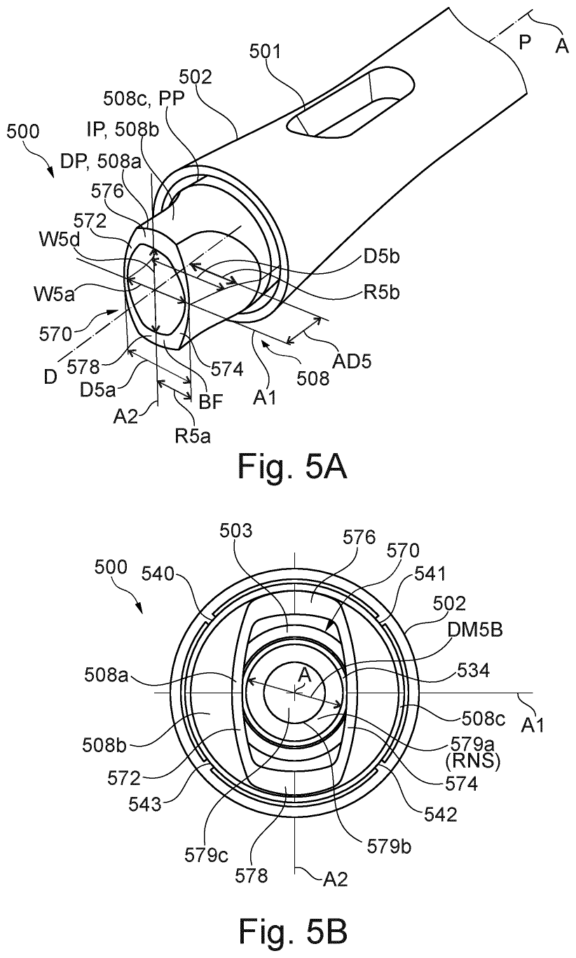

A needle cover spring 260 may be arranged within needle cover 208 biasing needle cover 208 distally D. Spring 260 may be arranged around distal tip of syringe 230. Syringe 230 may be hold within housing 202, e.g. using a central cylindrical portion that may be hold by ribs extending axially and radially, see e.g. similar ribs 540 to 543 and similar central housing part 503 as illustrated in FIG. 5B. However, in other embodiments, needle cover 208 may not be movable with regard to housing, e.g. spring 260 may be omitted. Moreover, housing 202 may be modified to comprise distal portion DP, intermediate portion IP and proximal portion PP as a distal device portion DDP.

A line L4a indicates the distal end of distal portion 202a of housing 202. Furthermore, a line L4b indicates the interface (touching region) between needle cover 208 and skin 220. Exemplary, a nail 421 of thumb 200h is illustrated. As is clearly visible in FIG. 4, width W3a of opening 370 is much smaller than e.g. diameter D3c thus enabling a narrow needle cover 208. Moreover, width W3a may be smaller than a diameter of barrel 232 of syringe 230 and even smaller than a maximum diameter of neck portion 236 (cone). A device using a rotationally symmetric needle cover or an essentially rotationally symmetric needle cover may remove the RNS though a grabber which may be attached to the entire circumference of the RNS. For a device using a rotationally asymmetric needle cover, it may be possible to remove the RNS through a design in which the grabber holds the RNS in two opposite places at the wider ends (sides) of the needle cover hole, and does not hold the RNS where the thin portions of the needle cover would interfere.

It is of course possible that a distance D4 between thumb 200h and index finger 200i is less than diameter D3c. In this case, thumb 200h and index finger 200i may be arranged closer to concave side faces 389a, 389b and may even touch these side faces 389a, 389b, especially during preparation of the injection. Preferably, the distance between thumb 200h and index finger 200i and/or the distance of these fingers 200h and 200i to line L4a are adapted such that device 200 does not interfere with these fingers during injection, e.g. when needle cover 208, NC moves axially, e.g. proximally, relative to housing 102 when housing 102 is pressed against skin 220.

An angle An4 may be included between an outer face of the side wall of intermediate portion 208b, IP and longitudinal axis A. Angle An4 may be within one of the ranges mentioned in the introductory part of the description above. Angle An4 may enable a free line of sight of the user to the injection site pass the distal border of housing 202, e.g. within the intermediate space between lines L4a and L4b, especially into an area within the projection of the distal border of distal portion 202a of housing 202 in the direction of longitudinal axis A to skin 220, i.e. oblique to the distal direction.

FIG. 5A illustrates a perspective view of a drug delivery device 500 according to a third embodiment. Drug delivery device 500 may be similar to device 100 or to device 200 mentioned above. Device 500 may comprise:
At least one optional drug window 501, e.g. similar to drug window 501,
A main housing part 502, e.g. similar to housing parts 102 and 202,
A needle cover 508 (actuating element or not),
A syringe (not shown in FIG. 5A, see FIG. 5B illustrating some parts of this syringe), e.g. similar to syringe 230.

Furthermore, device 500 may comprise all or some of the parts mentioned above for drug delivery device 100 or 200, e.g. a piston rod, a drive mechanism, etc.

Needle cover 508 may comprise:
A distal portion 508a, DP,
An intermediate portion 508b, IP, and
A ring portion 508c or proximal portion PP.

As is clearly visible needle shroud 508 has a comparable narrow distal portion 508a, DP, an intermediate portion 208b, IP, e.g. that becomes wider on its outside with increased distance to the distal portion DP, e.g. within at least one plane comprising longitudinal axis A, and a ring portion 508c or proximal portion PP that may have a circular outer diameter and that does not become wider with increased distance to the distal portion DP.

A distal opening 570 of needle cover (shroud) 508 may comprise,
A curved long first side wall 572,
A curved long second side wall 574,
A short third side wall 576 if its length is compared with the length of side wall 572, e.g. a straight side wall 576 that may be parallel to axis A1, and
A short fourth side wall 578 if its length is compared with the length of side wall 572, e.g. a straight side wall 578 that may be parallel to axis A1.

A width W5a of opening 570 along axis A1 may be much greater than width W3a valid for device 200 of the second embodiment. A width W5d of opening 570 along axis A2 may be greater than width W5a, e.g. by factor 1.2 but less than factor 2 or less than factor 1.5. Thus, opening 570 may be an oval opening that is different from a slit as formed e.g. by opening 370. As is described below in the description of FIG. 5B, it is possible to remove a rigid needle shield 759a, RNS (second needle cover in the claims) from the syringe and through opening 570.

There may be the following dimensions in a plane comprising axis A1 and longitudinal axis A:
A first (radial) distance R5a of a radially facing outer face of distal portion 508a to longitudinal axis A, e.g. measured along axis A1,
A second (radial) distance R5b of a radially facing outer face of intermediate portion 508b to longitudinal axis A, e.g. measured along an axis parallel to axis A1,
An axial distance AD5 between the positions on longitudinal axis A that are used for measuring radial distance R5a and radial distance R5b. Axial distance AD3 may be greater than 3 mm or greater than 5 mm.
A width W5a of distal opening 570, e.g. measured along axis A1, and
Dimension similar to dimensions mentioned for device 200, e.g. similar to diameter D3c.

Distance R5b may be greater than distance R5a because of the widening of needle cover 508. Similar relations between these dimensions may be true as mentioned above for relations between dimensions of device 200.

There may be the following dimensions in a plane comprising axis A2 and longitudinal axis A:
A width W5d of distal opening 570, e.g. measured along axis A2,
Dimension similar to dimensions mentioned for device 200, e.g. similar to radial distance R3d, radial distance R3e, diameter D3f.

Similar relations between these dimensions may be true as mentioned above for relations between dimensions of device 200, e.g. width W5d may be greater than width W5a.

Radial outer extensions D5a and D5b may be used instead of distances R5a and R5b. Radial outer extensions D5a is twice the radial distance R5a. Radial outer extensions D5b is twice the radial distance R5b.

FIG. 5B illustrates a view in the direction of longitudinal axis A of drug delivery device 500. FIG. 5B illustrates:
Axial/radial ribs 540 to 543, e.g. extending radially inwardly from central housing part 502,
A central part 503 of housing part 502, e.g. hold by axial/radial ribs 540 to 543,
A shoulder 534 of the syringe. The syringe may be hold within central part 503, e.g. using a separate syringe carrier or without usage of a separate syringe carrier,
A rigid needle shield 579a, RNS, e.g. a needle shield attached to a neck portion (e.g. cone) of the syringe.
An optional distal opening 579b of rigid needle shield 579a, and
A flexible and/or elastic insert 579c (boot) of rigid needle shield 579a, e.g. an insert that may allow sterile transport and sterile storage of the syringe.

Rigid needle shield 579a may have a maximum diameter DM5B within a cross section that is perpendicular to longitudinal axis A.

The smallest width W5a of opening 570 may be slightly greater than diameter DM5B, e.g. providing a circumferential clearing around rigid needle shield 579a along the whole circumference of rigid needle shield 579a. Width W5a may be greater than diameter DM5B by at most 5 percent of diameter DM5B. Thus, easy removal of rigid needle shield 579a from the syringe (see shoulder 534) and through opening 570 may be possible without contacting edges of opening 570 or with only contacting one edge. However, in other embodiments width W5a may be equal to maximum diameter DM5B or may be slightly less, e.g. less than 5 percent of width W5a. Thus, e.g. in cases in which rigid needle shield 579a is conical, e.g. slightly conical, only the proximal portion of rigid needle shield 579a will make contact to the edges of opening 570, e.g. to edges on opposite sides of opening 570.

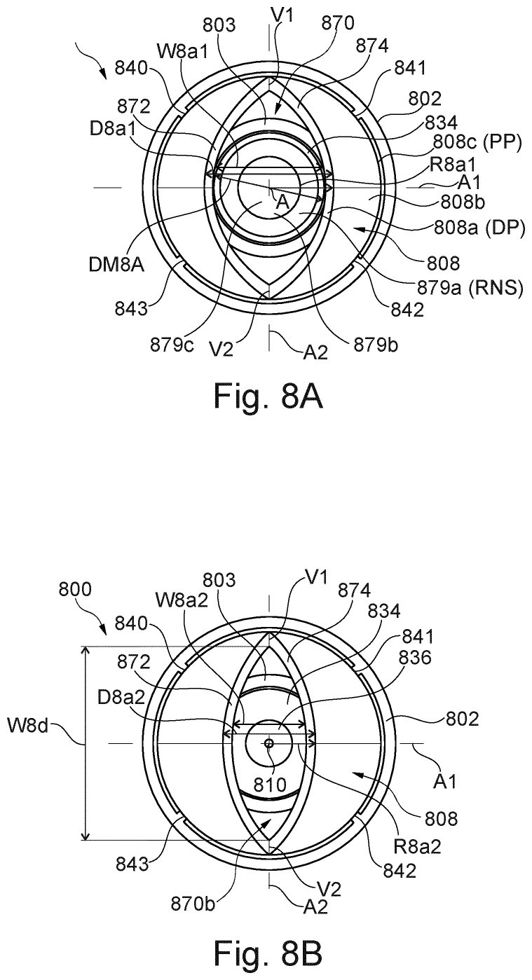

In a further embodiment, width W5a may be equal to or slightly less than a distal diameter, e.g. minimum diameter of rigid needle shield 579a. Thus, rigid needle shield 579a will be deformed in order to pass oval opening 570. It may be additionally or alternatively possible to deform the needle cover opening around the RNS diameter, although this may not be the primary embodiment. The needle cover opening could be held in position by, for example, the cap, which may be similar to the concept outlined in FIGS. 8A and 8B.

According to other embodiments, the outer shape of distal portion 508a, DP may be the same as mentioned above, or another shape, e.g. oval or rectangular. However, the distal opening may be rotationally symmetric, e.g. circular having a diameter that is equal to width W5a and its variants mentioned above.

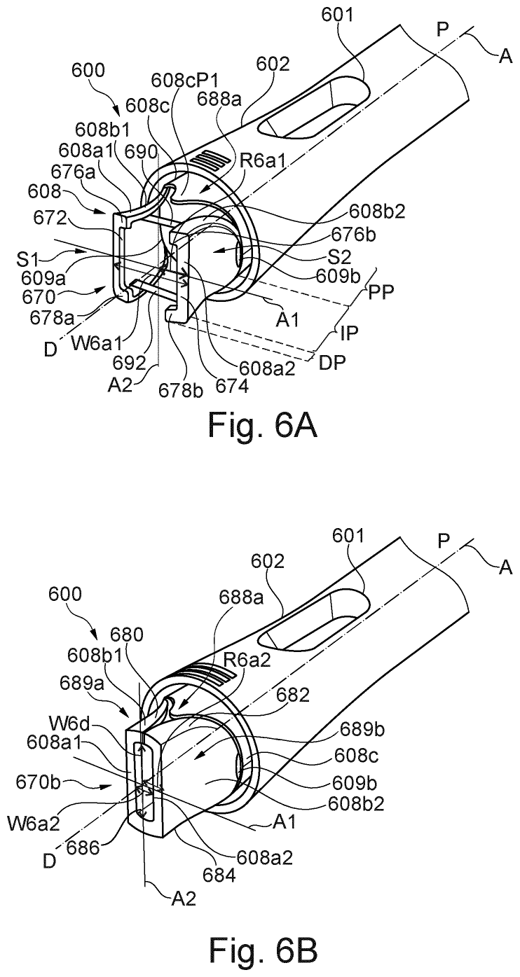

FIG. 6A illustrates a perspective view of a drug delivery device 600 according to a fourth embodiment with an open distal aperture 670. Device 600 may be similar to one of the devices 100 to 500. Device 600 may comprise:
At least one optional drug window 601,
A main housing 602 or housing part, and
A needle cover 608 that may be an actuating element or that may not be an actuating element of device 600.

Furthermore, device 600 may comprise all or some of the parts mentioned above for drug delivery device 100 to 500, e.g. a piston rod, a drive mechanism, etc.

Needle cover 608 may comprise.
At least two pivotable segments S1 and S2, and
A proximal portion 608c, PP, e.g. a ring portion or a cylindrical portion, see e.g. proximal portion 208c.

The pivotable segments S1 and S2 may be mechanically coupled to proximal portion 608c, PP, e.g. using coupling elements 609a, 609b that are described in more detail below. Pivotable segments S1 and S2 may be arranged around and extending along longitudinal axis A. At least in a closed position of pivotable segments S1 and S2, a needle may be encompassed from all lateral sides by the segments S1, S2 in order to prevent needle injuries and/or to hide the needle. There may be of course more than two segments arranged around longitudinal axis A.

The first pivotable segment S1 may comprise:
A first distal portion 608a1, e.g. corresponding to one half of distal portion 208a, and
A first intermediate portion 608b1, e.g. essentially corresponding to one half of intermediate portion 208b.

The second pivotable segment S2 may comprise:
A second distal portion 608a2, e.g. corresponding to the other half of distal portion 208a, and
A second intermediate portion 608b2, e.g. essentially corresponding to the other half of proximal portion 208b.

Thus, distal portion DP may comprise at least two distal portions 608a1, 608a2 or more than two distal portions, e.g. 3, 4 etc. Further, intermediate portion IP may comprise at least two intermediate portions 608b1, 608b2 or more than two intermediate portions, e.g. 3, 4 etc.

Coupling element 609a, 609b may comprise e.g. a film hinge that is formed integrally with and made of the same material as intermediate portions 608b1, 608b2 and proximal portion 608c, e.g. as a single part. Alternatively, other technical solutions may be used, e.g. two component injection molding, using a more flexible material for coupling elements 609a, 609b compared to the material used for intermediate portions 608b1, 608b2 and proximal portion 608c. Other hinge elements may be used as well, e.g. metal loops and/or hooks.

Ring portion 608c may comprise at least one or at least two distally protruding protrusions. One of these protrusions, e.g. protrusion 608cP1 is illustrated in FIG. 6A since it is located on an upper portion 688a of ring portion 608c. The other protrusion has a similar shape as protrusion 608cP1 but is arranged on the lower portion of ring portion 608 that is hidden in FIG. 6A by segment S2. These protrusions, e.g. 608cP1, are optional and the edges of segments S1 and S2 may be adapted to the shape of the protrusion, e.g. have a complementary shape.

An opening of needle cover 608 may have at least two or more than two states, e.g. an open state, see opening 670 as illustrated in FIG. 6A, and a closed state, see opening 670b as illustrated in FIG. 6B.

Opening 670 (open), 670b (closed) may comprise:
A long first side wall 672 on distal portion 608a1, e.g. a straight side wall, e.g. parallel to axis A2,
A long second side wall 674 on distal portion 608a2, e.g. a straight side wall, e.g. parallel to axis A2,
On distal portion 608a1, DP, a first segment 676a of a short third side wall, e.g. a straight segment 676a, e.g. parallel to axis A1,
On distal portion 608b1, DP, a second segment 676b of the short third side wall, e.g. a straight segment 676b, e.g. parallel to axis A1,
On distal portion 608a1, DP, a first segment 678a of a short fourth side wall, e.g. a straight segment 678a, e.g. parallel to axis A1, and
On distal portion 608b1, DP, a second segment 678b of short fourth side wall, e.g. a straight segment 678b, e.g. parallel to axis A1.

Axially directed edges 680 to 686, see FIG. 6B, may correspond to edges 380 to 386 mentioned above. Edges 680 and 686 may form borders of a first concave side face 689a, see e.g. FIG. 6B, forming e.g. an intermediate space for a finger pressing the skin, e.g. thumb and index finger. Edges 682 and 684 may form borders of a second concave side face 689a, see e.g. FIG. 6B, forming an intermediate space for a fingers pressing the skin, e.g. thumb and index finger, or at least allowing the finger to be placed closer to longitudinal axis A and needle passing opening, e.g. distal opening 670b, compared to the usage of a needle cover that has a distal portion DP having essentially the same outer diameter as proximal portion PP.

A first resilient element 690, e.g. a stretch band, a rubber band, an elastic band, etc., may be arranged between segments S1 and S2, e.g. coupled or fastened to the intermediate portions 608b1, 608b2, IP, e.g. close to edges 680 and 682 respectively. Thus, an extensible material may be used that is an elastic material which after releasing of the load returns to the undeformed initial state which was present before application of the load. First resilient element 690 may extend parallel to axis A1 in its expanded state. Similar, a second resilient element 692, e.g. a stretch band, a rubber band, an elastic band, etc. may be arranged between segments S1 and S2, e.g. coupled or fastened to the intermediate portions 608b1, 608b2, IP, e.g. close to edges 686 and 684 respectively. Second resilient element 692 may also extend parallel to axis A1 in e.g. its expanded state.

In the open (first) state of segments S1 and S2, a first radial distance R6a1 between radially outwardly facing outer face of distal portion DP and longitudinal axis A is again measured along axis A1. Radial distance R6a1 may have a first value in the open state of segments S1 and S2.

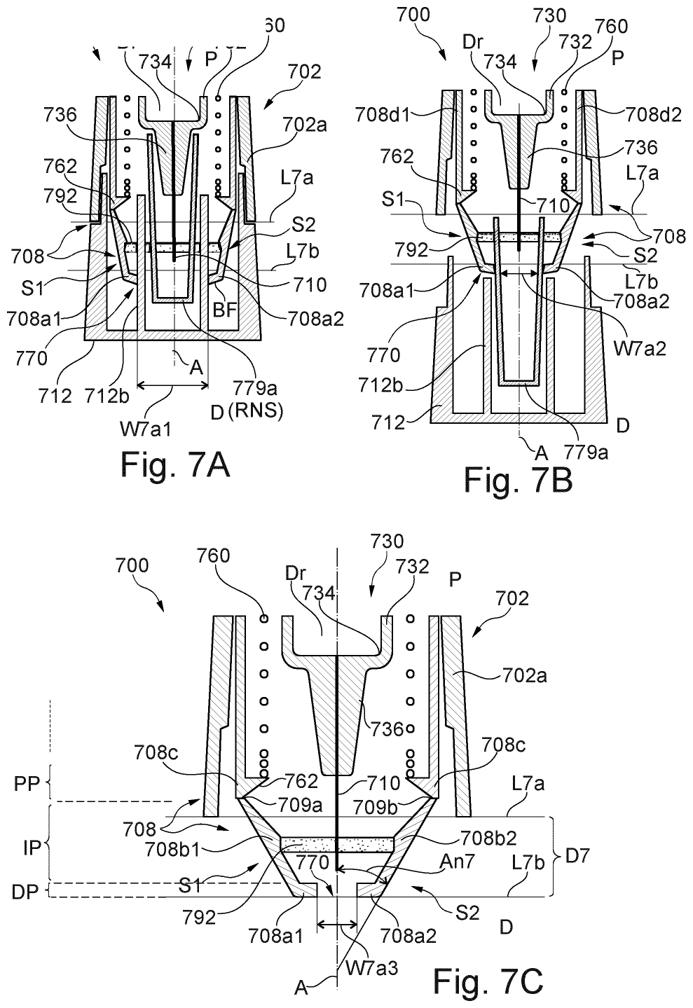

A width W6a1 of opening 670 extends in the open (first) state of segments S1 and S2 along first axis A1. Width W6a1 may be the same as an outer diameter of an insert of a cap of device 600, see e.g. similar insert 712b in FIG. 7A. The insert may be used to hold a rigid needle shield. Alternatively, width W6a1 may be the same as an outer diameter of a rigid needle shield, see e.g. similar rigid needle shield 779a as illustrated in FIG. 7A.

FIG. 6B illustrates a perspective view of drug delivery device 600 in a second state with closed segments S1, S2, e.g. in a closed state. In the closed (second) state, a first radial distance R6a2 between a part of radially outwardly facing outer face of distal portion DP and longitudinal axis A is again measured along axis A1. Radial distance R6a2 may have a second value in the closed state of segments S1 and S2. Radial distance R6a1 (first value) may be greater than radial distance R6a2 (second value), e.g. greater by at least factor 1.5, 2, 3 or 4.

A width W6a2 of opening 670b in closed (second) state of segments S1, S2 is measured along first axis A1. Width W6a1 may be greater than width W6a2, e.g. greater by at least factor 1.5, 2, 3 or 4. A width W6d of opening 670 measured along second axis A2 may be the same in both states of segments S1, S2, e.g. it may remain constant or unchanged.

Although opening 670 (open state), 670b (closed state) has a slit shape in the embodiment illustrated in FIGS. 6A and 6B, i.e. in device 600, other shapes may also be contemplated and used, e.g. a circular shape, a quadratic shape, etc.

Device 600 may comprise a needle cover 608 that is axially movable with respect to housing 602. A compression spring may be used to bias needle cover 608 distally, e.g. a spring that is similar to spring 260, see FIG. 4, or to spring 760, see FIGS. 7A to 7C.

Alternatively, needle cover 608 may be axially and/rotationally fixed to housing 602, e.g. there may be no needle cover spring 260, 760. Moreover, in other embodiments a distal device portion DDP may be used that is part of the housing 602, e.g. for devices that comprise a needle insertion and/or needle retraction mechanism which may move the syringe or another container axially relative to the housing.

FIG. 7A illustrates a longitudinal cross section of a drug delivery device 700 according to a fifth embodiment with an open distal aperture, e.g. distal opening 770 and a cap 712 and a rigid needle shield 779a in place. Drug delivery device 700 may comprise:
At least one optional drug window, see e.g. drug window 201, 501, 601, etc.,
A main housing part 702, see e.g. main housing part 102, 202, 502, 602, etc., and
A needle cover 708, e.g. used as an actuating element of device 700.

Housing part 702 may comprise distal part 702a. Furthermore, device 700 may comprise all or some of the parts mentioned above for drug delivery device 100 to 600, e.g. a piston rod, a drive mechanism, etc.

There may be a first segment S1, e.g. similar to segment S1 illustrated in FIGS. 6A and 6B. Furthermore, There may be a second segment S2, e.g. similar to segment S2 illustrated in FIG. 6A.

Segment S1 may comprise:
A first distal portion 708a1 of needle cover 708, e.g. similar to distal portion 608a1, and
A first intermediate portion 708b1 of needle cover 708, see FIG. 7C, e.g. similar to intermediate portion 608b1.

Segment S1 may comprise:
A second distal portion 708a2 of needle cover 708, e.g. similar to distal portion 608a2, and
A second intermediate portion 708b2 of needle cover 708, see FIG. 7C, e.g. similar to intermediate portion 608b2.

Further, needle cover 708 may comprise a ring portion 708c (proximal portion PP), e.g. similar to ring portion 608c.

Segments S1 and S2 may be pivotable relative to proximal portion 708, PP. Coupling elements 709a, 709b (film hinge etc.) may be used that are similar to coupling elements 609a and 609b.

An optional first arm 708d1 of needle cover 708 (needle shroud), see FIG. 7B, may extend proximally from ring portion 708c. An optional second arm 708d2 of needle cover 708 (needle shroud), see FIG. 7B, may also extend proximally from ring portion 708c. Alternatively, only one arm 708d1, 708d2 may be used for triggering of device 700. In other embodiments of device 700, device 700 may not be triggered using needle cover 708, e.g. arms 708d1 and 708d2 may be omitted.

As is illustrated in FIG. 7A, a needle 710 extends along longitudinal axis A from a cone portion 736 of a syringe 730 up to the intermediate portions 708b1, 708b2, PP but not up to the distal portions 708a1, 708a2, DP. This applies to the state illustrated in FIG. 7A as well as to the states illustrated in FIGS. 7B and 7C.

A cap 712 may comprise an outer wall, e.g. a first cylindrical wall, and an inner wall, e.g. a second cylindrical wall, e.g. having a smaller outer diameter that an inner diameter of the first cylindrical wall. The inner cylindrical wall may form an insert 712b. Insert 712b may hold a rigid needle shield 779a, RNS, e.g. using a metal part (not shown). Cap 712 may comprise a distal closing portion, e.g. a circular closing portion. The outer wall and the inner wall may extend proximally from the distal closing portion.

A syringe 730 may be arranged within a syringe retaining space of device 700, e.g. using a separate syringe carrier that may be a separate part from housing 702 and also a separate part from a central portion of housing part 702. FIG. 8B illustrates a central portion 803 that may be similar to the central portion of device 700. The syringe carrier and the syringe may be support in the central housing portion. Alternatively, no further syringe carrier may be used and syringe 730 may be hold directly within a central portion.

Syringe 730 may be aligned with its longitudinal axis to longitudinal axis A. Syringe 730 may comprise in the direction from its proximal end to its distal end an optional flange portion (not shown) having a maximal outer diameter of syringe 730a, e.g. a flange or flange portion (not shown) having flat portions on opposite lateral sides, a barrel portion 732, e.g. a cylindrical barrel portion 732, a shoulder 734 and a neck portion 736, e.g. comprising a conical shape. Shoulder 734 may connect barrel portion 732 and neck portion 736. Syringe 730 may be made of glass. Needle 710 may be an integral part of syringe 730.

A distal opening 770 of distal portions 708a, 708b may have a width W7a1, e.g. along an axis that is parallel to an axis that is similar to axis A1 illustrated in other figures, e.g. in FIG. 6B (closed state). Width W7a1 may have a value that is the largest value of all three states of device 700 illustrated in FIGS. 7A to 7C. Inner edges of distal portions 708a1 and 708a2 may abut to the outer face of insert 712b which may therefore define the value of width W7a1 in the state illustrated in FIG. 7A, e.g. width W7a1 may be equal to the outer diameter of insert 712b, especially at the abutting regions/points.

A line L7a indicates a plane that comprises the distal end of distal portion 702a of housing 702. A line L7b indicates a plane in which the skin of a patient will be arranged if bearing face BF is brought into contact with the skin but without activating device 700. As is apparent from FIG. 7A, distal portions 708a1 and 708a2 may be arranged distally from this latter plane, e.g. distally of line L7b.

There may be a distance D7 (see FIG. 7C) between lines L7a and L7b that may be in the range of 10 mm to 20 mm.

A needle cover spring 760 may be arranged around a distal portion of syringe 730, e.g. around neck portion 736 and/or around a distal portion of barrel portion 732. A distal end of spring 760 may be hold by a circumferential ledge 762 at the inner circumference of proximal portion 708c or by other appropriate means. A proximal end of spring 760 may bear against an inner part of housing 702, e.g. against a part that is arranged between outer wall of housing 702 and the central portion mentioned above. Spring 760 may be a compression spring that biases needle cover 708 distally.

Rigid needle shield 779a may be arranged around neck portion 736 and around needle 710 in close mechanical contact to both elements 736, 710 thereby forming a sterile protection for needle 710. Rigid needle shield 779a may be slightly tapered into the distal direction. Alternatively, there may be no tapering of rigid needle shield 779a.

A first resilient element, e.g. a stretch band, a rubber band, an elastic band or another appropriate element is not visible in FIGS. 7A to 7C but may be similar to resilient element 690 as illustrated in FIGS. 6A and 6B. The first resilient element may connect segments S1 and S2 at first outer edges of segments S1 and S2 or on other appropriate locations. A second resilient element 792, e.g. a stretch band, a rubber band, an elastic band or another appropriate element may connect segments S1 and S2 at second outer edges of segments S1 and S2 or on other appropriate locations. Both resilient elements, e.g. 792, may bias segments S1 and S2 radially inwards against insert 712b, e.g. against inner cylindrical wall of cap 712.

A radial distance of radially facing outer face of distal portions 708a1, 708a2 may be similar to distance R6a1 (see FIG. 6A) in the open state that is illustrated in FIG. 7A. This radial distance multiplied by factor two (2) may result in a distance (radial outer extension) that may be slightly greater than width W7a1.

FIG. 7B illustrates a longitudinal cross section along longitudinal axis A of drug delivery device 700 during removal of the cap 712 and of rigid needle shield 779a, RNS that may be attached to cap 712. There is a distance W7a2 between inner edges of distal portions 708a1, 708a2 in a second state (during removal of cap and RNS) that is illustrated in FIG. 7A. Again, distance W7a2 may be the width of opening 770 along axis A1 or along an axis that is parallel to axis A1. Width W7a2 may be smaller than width W7a1 due to the half-closed state illustrated in FIG. 7B.

Thus, in the second state, inner edges of distal portions 708a1 and 708a2 may abut to the outer face of rigid needle shield 779a, RNS which may therefore define the value of width W7a2 in the state illustrated in FIG. 7B, e.g. width W7a2 may be equal to the outer diameter of rigid needle shield 779a, RNS, especially at the abutting regions/points. The resilient elements, e.g. 792, may further bias segments S1, S2 radially inwards.

FIG. 7C illustrates a longitudinal cross section of drug delivery device 700 with the cap 712 and the rigid needle shield 779a, RNS removed. Therefore, segments S1 and S2 are in a closed state (third state), e.g. in a state that may be used during insertion of needle 710 into a skin and/or during injection of drug Dr stored within syringe 730.

In the closed state, an angle An7 may be included between an outer face of the side wall of intermediate portion IP and longitudinal axis A, e.g. between the side wall (e.g. outer face of side wall) of second intermediate portion 708b2 and the longitudinal axis A. Angle An7 may be within one of the ranges mentioned in the introductory part of the description above, e.g. within the range of 30 degrees to 60 degrees. Angle An7 may enable a free line of sight of the user to the injection site pass the distal border of distal portion 702a of housing 702, e.g. into the intermediate space between lines L7a and L7b, especially into an area within the projection of the distal border of distal portion 702a housing 702 in the direction of longitudinal axis A to a skin, i.e. in the distal direction D.

There may be a distance W7a3 between inner edges of distal portions 708a1, 708a2 in the third state (cap and RNS removed), e.g. the width W7a3 may be the width of opening 770 along axis A1.

A radial distance of radially facing outer face of distal portions 708a1, 708a2 may be similar to distance R6a2 (see FIG. 6B) in the closed state that is illustrated in FIG. 7C. This radial distance multiplied by factor two (2) may result in a distance (radial outer extension) that may be slightly greater than width W7a3.

Alternatively, needle cover 708 may be axially and/rotationally fixed to housing 702, e.g. there may be no needle cover spring 760. Moreover, in other embodiments a distal device portion DDP may be used that is part of the housing 702, e.g. for devices that comprise a needle insertion and/or needle retraction mechanism which may move the syringe or another container axially relative to the housing.

FIG. 8A illustrates a perspective view of a drug delivery device 800 according to a sixth embodiment with an open distal aperture 870 of a flexible and resilient needle cover 808. Drug delivery device 800 may comprise:
A main housing part 802, see e.g. housing parts 102 to 702 mentioned above,
A central part 803 of housing part 802, e.g. a cylindrical central part, and
A needle cover 808, e.g. a needle cover that may be used as an actuating element or not, see e.g. needle covers NC, 208 to 708 mentioned above.

Furthermore, device 800 may comprise all or some of the parts mentioned above for drug delivery device 100 to 700, e.g. a piston rod, see e.g. piston rod 104, a drive mechanism, see e.g. drive mechanism 106, etc.

Needle cover 808, NC may comprise:
A distal portion 808a, e.g. an elastic portion,
An intermediate portion 808b, e.g. an elastic portion, and
A proximal ring portion 808c, e.g. an elastic portion or a rigid portion.

If proximal portion 808c is made of a different material compared to the material of distal portion 808a and/or intermediate portion 808b a two component molding may be used or another appropriate technical solution.

Thus, an extensible material may be used for needle cover 800, NC that is an elastic material which after releasing of the load returns to the undeformed initial state which was present before application of the load. The amount of elasticity is chosen such that e.g. a rigid needle shield 879a may be removed comparably easily. However, the resiliency may still allow to use needle cover 808, NC for triggering of device 800 in some embodiments.

A syringe of device 800 may comprise a needle 810 (see FIG. 8B). Needle 810 may be an integral part of the syringe. The syringe may further comprise an optional flange (not shown), a barrel portion (not shown), a shoulder 834 (see FIG. 8B) and a neck portion 836 (see FIG. 8B).

Axial/radial ribs 840 to 843 may connect central portion 803 and housing 802, especially an outer wall of housing 802.

A distal opening 870 (open), 870b (closed) is formed in distal portion 808a. Thus, distal portion 808a may comprise:
A long first side wall 872, e.g. a curved side wall, and
A long second side wall 874, e.g. a curved side wall.

Side walls 872 and 874 may touch at vertexes V1, V2 without further sidewalls therein between. Alternatively, there may be a short third side wall, e.g. a straight or a curved side wall, and a short fourth side wall, e.g. a straight or a curved side wall, arranged between side walls 872 and 874 in distal portion 808a. As is mentioned below in more detail, vertexes V1, V2 may be arranged not only within the plane that comprises distal opening 870 but more proximally to distal portion 808a in other embodiments, e.g. thereby forming slits within intermediate portion 808b, IP. Other configurations are of course also possible.

A rigid needle shield 879a, RNS may be arranged within needle cover 808. Rigid needle shield 879a, RNS may be similar to rigid needle shield 579a or 779a mentioned above. Rigid needle shield 879a, RNS may comprise:
A proximal opening (not shown),
An optional distal opening 879b, e.g. a circular opening, and
A flexible insert 879c, e.g. an elastic insert (boot).

Side walls 872 and 874 may abut to the outer surface of rigid needle shield 879a, RNS in the state that is illustrated in FIG. 8A, e.g. in a state in which rigid needle shield 879a, RNS is still attached to the syringe of device 800. Due to the elasticity of the material of distal portion 808a and/or due to the elasticity of the material of the intermediate portion 808b, side walls 872 and 874 of distal portion 808a are biased inwards against rigid needle shield 879a, RNS. Rigid needle shield 879a, RNS may have a diameter DM8A at the abutting portions. Therefore a value of width W8a1 of opening 870 may be equal to the value of diameter DM8A. Even if rigid needle shield 879a, RNS is slightly conical it remains easy to remove rigid needle shield 879a, RNS from device 800 due to the elasticity of needle cover 808, NC.

There may be a first distance R8a1 in the open (first) state. First distance R8a1 may have a first value and may be measured along a first axis A1. A radial outer extension D8a1 of distal portion 808a may have a value that is twice the value of distance R8a1. Radial outer extension D8a1 may also be measured along axis A1. A width W8a1 of opening 870 in the open (first) state may be measured along the first axis A1

FIG. 8B illustrates a perspective view of drug delivery device 800 a with a narrow (half closed) distal aperture or opening 870b after rigid needle shield 879a, RNS has been removed.

Due to the resiliency of the elastic material of needle cover 808, NC the first distance is changed to a distance R8a2 in the narrow (second) state, e.g. distance R8a2 that is similar to first distance R2a1 has a second value in the second state that is smaller than the first value (distance R2a1). A value of a radial outer extension D8a2 that is similar to radial outer extension D8a1 is also reduced compared to a value of radial outer extension D8a1.

A width W8a2 of opening 870 in the narrow (second) state and along first axis A1 is smaller than the corresponding width W8a1. A width W8d of opening 870 in both states and along second axis A2 is the same in both states of device 800.

There may be a radial distance similar to radial distance R3b, see FIG. 3, to an outer surface of intermediate portion 808b, IP. This radial distance of intermediate portion 808b, IP may also be decreased between the two states illustrated in FIGS. 8A and 8B, e.g. there may be a transversal movement of the wall of intermediate portion 808b, IP radially inwards or inwards.

Vertexes V1, V2 may be arranged in the same plane in which distal portion 808a is arranged. Alternatively, there may be other possibilities to arrange vertexes V1 and V2, e.g. within intermediate portion 808b, IP or even at proximal portion 808c, PP. Vertexes at a plane other than the distal plane may be possible, e.g. subject to detailed design.

Device 800 may comprise a needle cover 808 that is axially movable with respect to housing 802. A compression spring may be used to bias needle cover 808 distally, e.g. a spring that is similar to spring 260, see FIG. 4, or to spring 760, see FIGS. 7A to 7C.

Alternatively, needle cover 808 may be axially and/rotationally fixed to housing 802, e.g. there may be no needle cover spring similar to spring 260, see FIG. 4 or to spring 760, see FIGS. 7A to 7C. Moreover, in other embodiments a distal device portion may be used that is part of the housing 802, e.g. for devices that comprise a needle insertion and/or needle retraction mechanism which may move the syringe or another container axially relative to the housing.

Thus, needle cover 808 may be movable relative to housing 602, e.g. using a needle cover spring, similar to spring 260, 760 mentioned above. It may be possible to use a material or geometry that is not isotropic, i.e. a material or geometry which is sufficiently flexible in the direction relevant for clearing the RNS, that can flex outward radially, but that is strong or comparably stronger if compressed in the axial direction, e.g. if compared with the compressibility in the direction relevant for clearing the RNS. Alternatively, needle cover 808 may be axially and/rotationally fixed to housing 802 or it may be part of the housing, e.g. there may be no needle cover spring similar to spring 260, 760.

Spoken with other words, a drug delivery device, e.g. 100 to 800, may comprise:
a housing or housing part, e.g. 102 to 802, extending along a longitudinal axis A of the drug delivery device 100 to 800, and
a distal device portion DDP,
wherein the distal device portion DDP may comprise a distal portion DP, a proximal portion PP and at least one intermediate portion IP,
wherein the distal portion DP may comprise a distal opening, e.g. 370, allowing passage of a needle, e.g. 210, and a bearing surface BF,
wherein the bearing surface BF may be configured to be pressed against the skin, e.g. 220, of a patient during injection of a drug Dr,
wherein the intermediate portion IP may comprise at least one wall portion extending proximally from the distal portion DP to the proximal portion PP,
wherein in at least one state of the drug delivery device 100 to 800, a first radial outer extension, e.g. D3a, of the distal portion DP in a region axially adjoining the bearing surface BF may be smaller than a second radial outer extension, e.g. D3b, in the intermediate portion IP, and
wherein an outer face of the intermediate portion IP may be inclined, see e.g. angle An4, An7 relative to the longitudinal axis A in a cross section along the longitudinal axis A, e.g. a cross section including the longitudinal axis A.

Angle An4, An7 may be in the range of 15 degrees to 75 degrees in the range of 30 degrees to 60 degrees or in the range of 40 degrees to 50 degrees.

Thus, a design of a needle cover NC for a drug delivery device 100 to 800 is provided with enhanced usability due to e.g. a narrow distal opening 370 to 870, 870b, etc.

A drug delivery device 100 to 800 is provided, e.g. with a locking needle cover, for post-injection needle safety, whereby the needle cover NC may be narrower in one direction, allowing for easy manipulation of the local skin by the user.

It is noted that many current drug delivery devices with needle covers may only have circular openings for means of clearing syringe or cartridge components and providing a stable base for injection. However, the proposed design covers a range of implementations particular to e.g. a narrow needle cover, allowing e.g. easy pinching of the skin during injection.

Implementations may include:
An autoinjector compatible with a Pre Filled Syringe (PFS), whereby the needle cover geometry may be narrower at the distal end while clearing the PFS needle shield, e.g. RNS.
An autoinjector compatible with a PFS, whereby the needle cover NC may change form on the removal of the PFS needle shield, e.g. RNS.
A drug delivery device, e.g. 100 to 800, compatible with a cartridge or other medicament container with e.g. a narrow needle shield.

One embodiment of the previous design may relate to a design where the needle shield/cover NC contains elements in tension and flexible areas (e.g. hinges) such that when stored in conjunction with a cap and/or PFS shield, the needle cover NC is held in an open position.

Once the cap, e.g. 112, 712, and/or PFS shield, e.g. RNS, is removed, the needle cover NC may close into a form which affords protection from needle stick injury while also offering a narrow opening such that the user can pinch the skin during injection.

Also considered may be an embodiment in which the ‘tension’ and ‘hinge’ elements are intrinsic to the material and/or geometry such that the aperture of the needle cover NC collapses to a narrower position on removal of the cap, e.g. 112, 712, and/or PFS shield, e.g. RNS, see e.g. FIGS. 8A and 8B.

The solution illustrated in FIGS. 5A, 5B may have tight clearances or no clearances to the RNS (rigid needle shield) and may therefore require a different cap and grabber concept compared to device concepts using essentially rotationally symmetric concepts of needle cover. However, the exterior design and use steps may be largely retained.

The elastically closable or partially closable solution illustrated in FIGS. 6A, 6B, 7A, 7B, 7C may be compatible with essentially rotationally symmetric cap concept which may require only small changes in cap geometry.

The design in FIGS. 8A, 8B may have tight clearances or no clearances to the RNS and may therefore require a different cap and grabber (element for holding the RNS within the cap, e.g. made of metal) concept compared to an essentially rotationally symmetric cap. However, the exterior design and use steps could be largely retained.

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure, systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. Further, it is possible to combine embodiments mentioned in the first part of the description with examples of the second part of the description which relates to FIGS. 1A to 8B.

1-20. (canceled)

21. A drug delivery device comprising:
a housing extending along a longitudinal axis of the drug delivery device; and
a distal device portion,
wherein the distal device portion comprises a distal portion, a proximal portion and at least one intermediate portion,
wherein the distal portion comprises a distal opening allowing passage of a needle and a bearing surface,
wherein the bearing surface is configured to be pressed against the skin of a patient during injection of a drug,
wherein the intermediate portion comprises at least one wall portion extending proximally from the distal portion to the proximal portion,
wherein in at least one state of the drug delivery device, a first radial outer extension of the distal portion in a region axially adjoining the bearing surface is smaller than a second radial outer extension in the intermediate portion, and
wherein at least a region of an outer face of the intermediate portion is inclined relative to the longitudinal axis in a cross section along the longitudinal axis.

22. The drug delivery device according to claim 21, wherein in a cross section including or along the longitudinal axis, the outer face of the intermediate portion forming an angle with the longitudinal axis, the angle being within a range of 15 to 75 degrees.

23. The drug delivery device according to claim 21, wherein the distal device portion is at least a portion of a needle cover coupled to the housing,
wherein the needle cover is a part that is manufactured separate from the housing,
wherein the needle cover is movable relative to the housing, and
wherein the needle cover does not have direct mechanical contact to a drug container and/or to a needle of the drug delivery device.

24. The drug delivery device according to claim 21, wherein the distal portion and at least a part of the intermediate portion are accessible on the exterior of the drug delivery device at least before the injection of the drug and/or before insertion of a needle of the drug delivery device.

25. The drug delivery device according to claim 21, wherein the distal portion has a rotationally asymmetric outer shape with respect to a rotation around the longitudinal axis, and/or
wherein the distal opening has a rotationally asymmetric shape with respect to a rotation around the longitudinal axis.

26. The drug delivery device according to claim 21, wherein the first radial outer extension and the second radial outer extension are the same in all operating states of the drug delivery device,
wherein the distal device portion is a first needle cover or is comprised in a first needle cover,
wherein the drug delivery device comprises a second needle cover,
wherein the second needle cover has a maximal outer diameter in a cross section perpendicular to the longitudinal axis, and
wherein the distal opening has a first width along the first axis, and wherein the first width is greater than the maximal outer diameter.

27. The drug delivery device according to claim 21,
wherein the distal device portion is a first needle cover or is comprised in a first needle cover,
wherein the needle cover comprises at least two segments,
wherein a first segment of the at least two segments comprises a first part of the distal portion and a first part of the intermediate portion,
wherein a second segment of the at least two segments comprises a second part of the distal portion and a second part of the distal portion,
wherein the first part of the intermediate portion is pivotable coupled to the proximal portion of the needle cover via a first coupling element,
wherein the second part of the intermediate portion is pivotable coupled to the proximal portion of the needle cover via a second coupling element,
wherein in an open state of the at least two segments, the first radial outer extension has a first value, and
wherein in a closed state of the at least two segments, the first radial outer extension has a second value.

28. The drug delivery device according to claim 27, wherein the drug delivery device comprises at least one resilient element that biases the at least two segments to pivot into the closed state,
wherein the at least one resilient element is attached to the first segment and to the second segment, and
wherein the at least one resilient element is or comprises a tension element that applies a tension force to the at least two segments.

29. The drug delivery device according to claim 28, wherein the at least one resilient element is arranged inside the needle cover.

30. The drug delivery device according to claim 21, wherein the first coupling element and/or the second coupling element is formed integrally with the proximal portion of the needle cover.

31. The drug delivery device according to claim 21, wherein the needle cover is a first needle cover,
wherein the drug delivery device comprises a second needle cover, and
wherein in the open state, at least one of the first section and the second section abuts to the second needle cover or to an insert of a cap that holds the second needle cover.

32. The drug delivery device according to claim 21,
wherein at least one of the intermediate portion and the distal portion comprises an elastic material,
wherein the elastic material is configured to allow decreasing of the first radial outer extension,
wherein in an open state of the distal opening the first radial outer extension has a first value, and
wherein in a narrow state of the distal opening the first radial outer extension has a second value.

33. The drug delivery device according to claim 32,
wherein the distal device portion is a first needle cover or is comprised in a first needle cover or wherein the distal device portion is part of the housing,
wherein the drug delivery device comprises a second needle cover,
wherein in the open state, the distal portion abuts to the second needle cover or to an insert of a cap of the drug delivery device, and
wherein the insert of the cap supports and/or holds the second needle cover.

34. The drug delivery device according to claim 21, comprising at least one compression spring,
wherein the at least one compression spring is configured to bias a needle cover distally, and
wherein the needle cover comprises the distal device portion.

35. The drug delivery device according to claim 21, comprising a cap,
wherein the cap is rotationally asymmetric with respect to the longitudinal axis in order to adapt the cap to a rotational asymmetry of the distal device portion, an outer shape of the distal portion, an outer shape of the intermediate portion and an inner shape of the intermediate portion.

36. The drug delivery device according to claim 21, wherein the housing is provided to retain and/or retains a medicament container in its interior, wherein a medicament is arranged in the medicament container.

37. The drug delivery device according to claim 21, wherein the distal device portion is a distal portion of the housing and/or wherein the distal device portion is not movable relative to the housing.

38. The drug delivery device according to claim 21, wherein the distal device portion is a first needle cover or is comprised in a first needle cover, wherein the needle cover comprises at least two segments and at least one resilient element that biases the at least two segments, wherein the at least one resilient element is arranged inside the needle cover.

39. The drug delivery device according to claim 21, wherein the drug delivery device is an autoinjector.

40. A method comprising:
dispensing a medicament with the drug delivery device, wherein the drug delivery device comprises:
a housing extending along a longitudinal axis of the drug delivery device; and
a distal device portion,
wherein the distal device portion comprises a distal portion, a proximal portion and at least one intermediate portion,
wherein the distal portion comprises a distal opening allowing passage of a needle and a bearing surface,
wherein the bearing surface is configured to be pressed against the skin of a patient during injection of a drug,
wherein the intermediate portion comprises at least one wall portion extending proximally from the distal portion to the proximal portion,
wherein in at least one state of the drug delivery device, a first radial outer extension of the distal portion in a region axially adjoining the bearing surface is smaller than a second radial outer extension in the intermediate portion, and
wherein at least a region of an outer face of the intermediate portion is inclined relative to the longitudinal axis in a cross section along the longitudinal axis.
